(19) United States
(12) Patent Application Publication
(10) Pub. No.: US 2015/0307843 A1
(43) Pub. Date: Oct. 29, 2015

(54) METHOD TO OBTAIN HEMATOPOIETIC STEM CELLS USING THREE-DIMENSIONAL INDUCING SYSTEM

(71) Applicant: Zhejiang University, Hangzhou (CN)

(72) Inventors: He HUANG, Hangzhou (CN); Yulin XU, Hangzhou (CN); Xiaohong YU, Hangzhou (CN)

(21) Appl. No.: 14/562,230

(22) Filed: Dec. 5, 2014

(30) Foreign Application Priority Data
Apr. 27, 2014 (CN) 201410170829.6

(51) Int. Cl. C12N 5/0789

(52) U.S. Cl. CPC C12N 5/0647; C12N 2506/45; C12N 2501/20; C12N 2506/02; C12N 2501/22

(57) ABSTRACT

The invention relates to a method of acquiring hematopoietic stem cells (HSCs) by means of three dimensional inducing system with high efficiency—which makes use of three-dimensional cell culture matrix or cell culture scaffolds, such as three-dimensional cell culture system which is made from hydrogel, seaweed and other materials, and/or associating stromal-cell including bone marrow cell, mouse bone marrow cell line OP9, OP9DL1 and so on, and/or associating multiple factors including mesoderm inducible factor, hematopoietic growth factor etc which can induce pluripotent stem cells to differentiate into hematopoietic stem cells. The invention sets up a new method of acquiring hemopoietic stem cells, establishing a system that induces pluripotent stem cells to differentiate into hemopoietic stem cells with high efficiency by means of utilizing dimensional inducing system and/or associating stromal-cell including bone marrow cell and/or multiple factors for the first time, which provide a theoretical basis and technology platform for acquiring clinical available hematopoietic stem cells, and expand additional methodologies and ideas in fields of disease mechanism exploring, drug screening etc for hematopoietic cells from pluripotent stem cells.

### FIELD OF TECHNOLOGY

The present invention relates to biotechnology field, which involves a method of utilizing three-dimensional inducing system to obtain hematopoietic stem cells (HSCs).

### BACKGROUND OF THE INVENTION

The occurrence and development of hematopoiesis is a complex process. It starts at the second weekend since the formation of human embryo. At this time, extraembryonic mesoderm cells, locating in ovarian cystic wall, which are undifferentiated and able to self-renewal, make up blood islands and become the original human hematopoietic center. Starting from the sixth week after the formation of human embryo, hematopoietic stem cells (HSCs) generated by blood islands migrate to liver with bloodstream, plant therein and the liver hematopoietic period begins. In the third month since the formation of human embryo, long bone marrow begins to be hematopoietic. In the eighth month, bone marrow hematopoietic activity develops highly, at this time, liver and spleen hematopoietic function declined and marrow is turning to be the hematopoietic center. HSCs in marrow are adult stem cells with self-renewal ability and multi-differentiation potential, which are the source of all hematopoietic cells and immune cells in human blood system, playing an important role in maintaining normal function of hematological system and immune system. The percent of HSCs in marrow is very low. As the age grows after adolescence, human marrow starts to fade and is replaced by adipose tissue, the number of HSCs gradually decreases, resulting in declining of diversified functions of human body including immune function. Many diseases concerned with blood and immune system are closely related to whether the function of HSCs is normal or not. Now, HSCs have been widely used in treating a variety of diseases including malignant or benign tumour of hematological system, such as acute myelocytic leukemia, chronic granulocytic leukemia (CGL), aplastic anemia, thalassanemia etc, and immune system disorders, such as partly autoimmune disorder, severe combined immunodeficiency disorder and partly solid tumor etc. Older people with poor blood circulation or hypoimmunity may benefit from HSC transplantation in the future. HSC transplantation has broad clinical application prospects, and cells of which become a kind of tissue stem cells which are used most widely and are the most technologically mature ones, HSC transplantation is a successful profile of regenerative medicine.

Hematopoietic stem cell transplantation requires hematopoietic stem cells which are matched with major histocompatibility complex (MHC) or human leukocyte antigen (HLA). According to the China Bone Marrow Bank data (by Nov. 30, 2013), as many as 34,450 patients have applied for queries while only 3863 people donate hematopoietic stem cells, so many patients who need cell type matching often lost the best treatment opportunity during the process of waiting. At present, allogeneic HSC transplatation is the main method of cell transplantation, but it always leads to a series of fatal transplantation complications such as graft-versus-host disease due to immunologic rejection. Although umbilical blood can be substituted for adult HSCs for the use of transplantation, HSCs in umbilical blood are too few to meet the adults' demand for HSCs in transplantation. HSC is a kind of adult stem cell with extremely low number, and how to acquire abundant functional HSCs for the purpose of clinical use, is the primary problem of carrying out clinical treatment and research concerning HSCs at present.

Human pluripotent stem cells, including embryonic stem cells and induced pluripotent stem cells, have the ability to proliferate massively and chronically in the condition of appropriate in vitro culturation, and have the potential of differentiating into all cell types including hemopoietic stem cells which required by human body, which is a method to resolve source issue of HSCs. Hence, acquiring HSCs by means of induction and differentiation of human pluripotent stem cells has an important role in clinical application. At present, the embryoid body method and stromal cells co-culture method, induced factor method, combination of stromal cell and induced factor method and other methods are commonly used ways to induce pluripotent stem cells to induce pluripotent stem cells to differentiate into hematopoietic stem cells. The stromal cells are closely related to hematopoietic environment, including mice bone marrow stromal cells OP9 or S17, mouse aorta-gonad-mesonephros stromal cell AGM, mouse yolk sac endothelial cell line C166, human or mouse embroynic liver cell, modified stromal cell line OP9-DL1, etc. The inducing factors involved generally are mesoderm inducing factor as BMP4, PD98059, and hematopoietic and endothelial cells factors as SCF, FLT3L, VEGF, IL-3, IL-6, VEGF, etc. After a period of time (usually about 2-3 weeks) of co-culturing or induction, the percentage of the functional hematopoietic stem cells remains very low, the proportion of the cells expressing the early hematopoietic stem cell surface markers CD34+CD38− accounts for only 0.1%-2%. In function, although these cells can form part of the hematopoietic colony, and generate erythroid progenitor cells, myeloid progenitor cells, macrophages, etc., no myeloid and lymphoid cells can be obtained simultaneously. How to efficiently induce human pluripotent stem cells to differentiate into functional hematopoietic stem cells is the main bottleneck concerning clinical applications of pluripotent stem cells, and it also has very important significance for in vitro study of human hematopoietic development.

### SUMMARY OF THE INVENTION

The purpose of the invention is to provide a method of utilizing three-dimensional inducing system to obtain HSCs, which is realized by the following technical schemes: collect pluripotent stem cells by mechanical method, then dispose three-dimensional cell culture materials such as hydrogels, seaweed and nanomaterials according to manufacturers' instructions, and/or associating stromal cells, including marrow cells (human or mouse), mouse myeloid cell lines OP9, OP9-DL1 or other relevant stromal cells which have the ability to induce differentiation, and/or associating relevant small molecule materials, including mesoderm inducing factors BMP4, stem cell growth factor (SCF), Flt3L, vascular endothelial growth factor (VEGF), thrombopoietin (TPO), prostaglandin 2 (PGE2) and so on. After 10-14 days of induction and culture, HSCs are analyzed.

HSCs are obtained by the differentiation of human pluripotent stem cells, which include human embryonic stem cells or induced pluripotent stem cells. Said induced pluripotent stem cells are the specified induced pluripotent stem cells which are acquired through reprogramming by means of inducing human somatic cells such as fibroblasts, bone marrow mesenchymal stem cells (BMMSCs) and so on. The inducing process utilizes retrovirus, lentivirus, adenovirus or sendai virus which contains transcription factors like Oct4, Sox2, Klf4 and/or c-Myc to get the specified induced pluripotent stem cells. Said human embryonic stem cells are human embryonic stem cell lines as H1 and H9 which can be possibly acquired through commercial approach.

Said three-dimensional cell culture materials include multiple three-dimensional culture matrix or cell culture scaffolds, such as all kinds of hydrogels, seaweeds, nanomaterials and other three-dimensional cell culture materials. Furthermore, relevant hematopoietic factors including hematopoietic cytokine factors such as SCF, Flt3L, IL-3, IL-6, and PGE2, are used to amplify the obtained HSCs further. On the basis of obtained HSCs, SCF, IL-3, IL-6, ILG-7, IL-2, GM-CSF, EPO, TPO, etc., can be utilized to induce the whole system to differentiate into multiple pedigrees.

Detection of the proportion of hematopoietic stem cells by flow cytometry: In the process of induction and amplification, detect the expression of surface maker CD34 on HSC cells by the use of flow cytometry technical detection system. Measuring the potential of colony-forming units (CFU) generated from HSCs by the semisolid methylated cellulose culture: disperse the obtained HSCs into single cells, seed them in the semisolid methylated cellulose culture systems which include SCF, GM-CSF, IL-3, IL-6, TPO, EPO and other factors. Petri dish used must be of low adhesion and the culturation should be lasted for 2 weeks or so. Observe the generation of CFU in morphology and count.

This present method characterized its property by utilizing three-dimensional inducing system and/or associating stromal cell lines, and/or associating mesoderm inducing factors, hematopoietic factors and so on to induce pluripotent stem cells to differentiate into HSCs with high efficiency. The method would potentially provide clinic with reliable source of HSCs and positively promotes pluripotent stem cell-derived HSC clinical application. The invention sets up a new method to obtain HSCs, and for the first time sets up a system utilizing three-dimensional induction system and/or associating marrow cells and other stromal cells and/or associating multiple factors to induce pluripotent stem cells to differentiate into HSCs, which provides a theoretical basis and technology platform for acquisition of clinical available hematopoietic stem cells, and explores additional methodologies and ideas for the application of pluripotent stem cell-derived hematopoietic cells in fields like disease mechanism investigation, drug screening and so on.

### BRIEF DESCRIPTION OF THE DRAWINGS

The illustrations in the invention are represented three-dimensional induction system concerning hydrogels associating, mice bone marrow, OP9DL1 and cell factors.

FIG. 1. The scheme for three-dimensional induction and differentiation of HS Cs originated from pluripotent stem cells.

FIG. 2. The scheme for three-dimensional inducing and differentiating system of HSCs originated from pluripotent stem cells associating with cytokine.

FIG. 3. The cellular morphology in the first day of cultural in three-dimensional inducing and differentiating system of HSCs (4×).

FIG. 4. The cellular morphology during the 10th day to the 14th day of cultural in three-dimensional inducing and differentiating system of HSCs (4×).

FIG. 5. The grape-like cell clones appeared during the 10th day to the 14th day of cultural in three-dimensional inducing and differentiating system of HSCs (20×).

FIG. 6. The cellular morphological changes taken place in the 28th day of cultural in three-dimensional inducing and differentiating system of HSCs (4×).

FIG. 7. The T-like cells appeared in the 28th day of cultural in three-dimensional inducing and differentiating system of HSCs (20×).

FIG. 8. The cellular morphology of hemopoietic-like colonies appeared in the 28th day of cultural in three-dimensional inducing and differentiating system of HSCs (10×).

FIG. 9. The cellular morphology of hemopoietic-like colonies appeared in the 28th day of cultural in three-dimensional inducing and differentiating system of HSCs (10×).

FIG. 10. The measurement of total human cell number (detected by antibody TRA-1-851) and the ratio of CD34 positive cells after 10-14 days culturing in three-dimensional inducing and differentiating system.

FIG. 11. The proportion of CD34+ cells obtained in each group of different three-dimensional inducing and differentiating systems after 10-14 days of culture.

FIG. 12. CD34+ cells after 10 days' sorting from hemapoietic stem cells in three-dimensional inducing and differentiating system by CD34+ cell magnetic bead separation technology (20×).

FIG. 13. The proportion of CD34+ cells detected after application of flow cytometry technology to three-dimensional inducing system.

FIG. 14. The obtained hematopoietic colony granulocyte colony, granulocyte/macrophage arisen from human CD34+ cells cultured with methylated cellulose, which were derived from hematopoietic stem cells in three-dimensional inducing and differentiating system (4×).

FIG. 15. The obtained hematopoietic colony granulocyte colony, granulocyte/macrophage arisen from human CD34+ cells cultured with methylated cellulose, which were derived from hematopoietic stem cells in three-dimensional inducing and differentiating system (4×).

FIG. 16. The obtained hematopoietic colony erythroid colony, mixed lineage including erythroid/granulocyte/macrophage/megakaryocyte colonies arisen from human CD34+ cells cultured with methylated cellulose, which were derived from hematopoietic stem cells in three-dimensional inducing and differentiating system (4×).

### DETAILED DESCRIPTION

This invention combines drawings and examples for further explanation.

### Example 1

Use three-dimensional inducing system to induce pluripotent stem cells to differentiate into hematopoietic stem cells.

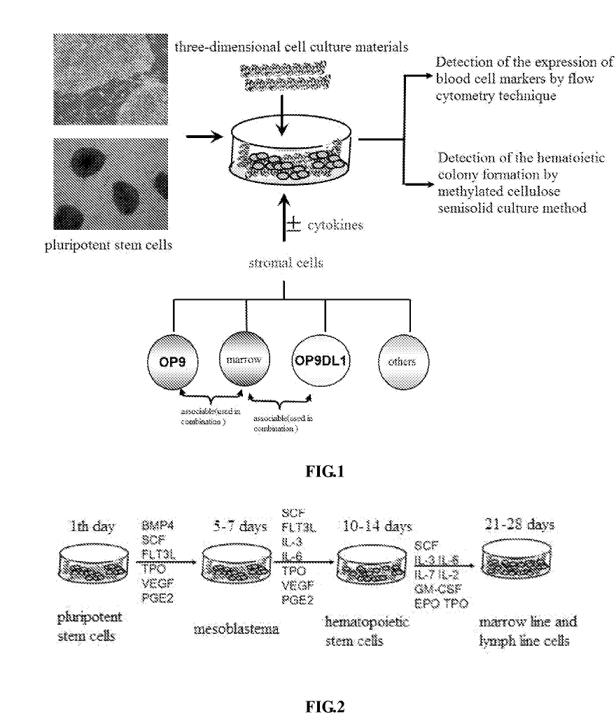

The invention provides a method of inducing pluripotent stem cell to differentiate into hematopoietic stem cells which makes use of three-dimensional cell culture materials, including hydrogels, seaweeds and other materials, and/or associating bone marrow stromal cell, and/or associating mouse bone marrow cell line OP9, or OP9DL1, and/or associating multiple factors including mesoderm induction factor, and hematopoiesis related factor. Detailed schemes are as shown in FIG. 1 and FIG. 2.

1. All the materials and reagents can be obtained from commercial way if no special instructions are indicated.

2. Culture, proliferation, passage of pluripotent stem cells

\textcircled{1} The preparation of trophoblast MEF (from Sidansai Biotechnology Company): processed according to conventional methods.
\textcircled{2} Thaw embryonic stem cells and pluripotent stem cells, seed them in trophoblast which has been treated with mitomycin C, cultivate them by means of specialized medium for pluripotent stem cells, the ingredients of culture medium include DMEM/F12, 20% KnockOut Serum Replacement (KSR), 2 mML derivatives of glutamine (GlutaMAX), 0.1 mM β-mercaptoethanol, 1% nonessential amino acids (NEAA), 50 U/mL penicillin, 50 mg/mL streptomycin and 4 ng/mL basic fibroblast growth factor (bFGF), cultured in an incubator set at 37° C., 5% CO₂. Change medium and observe the growth status of clones every day. When Clones grow to a certain size, digest by collagenase IV, passage or set aside.

3. Preparation for stromal cells \textcircled{1} Preparation of mouse bone marrow cells: Purchased mice (species, age and gender are not concerned), killed by broken. Soak them in 75% alcohol for 5-8 minutes and take them into a sterile room. Fix it on dissecting table, hand with a high temperature sterilized surgical instruments, such as scissors, surgical tweezers, cut open leg skins, remove the whole leg and place it in sterile saline. Then take them to super clean bench, remove the leg muscles. Cut open the ends of the bone, withdrawing saline with 1 ml syringe, flush the marrow cavity. The bone marrow is then collected by centrifugation, lyse red blood cell for 5-7 minutes by erythrocyte lysate. Saline wash 2-3 times. Finally the cells are collected and set aside.

\textcircled{2} Preparation of human bone marrow cells:
With the consent of volunteers, after they sign an informed consent form, take out 1-2 ml iliac bone marrow. Bring it into the sterile room, lyse red blood cells for 5-7 minutes with erythrocyte lysate. Saline wash 2-3 times, the cells are finally collected and set aside.
\textcircled{3} Preparation of placental cells. With the consent of their families, sign an informed consent form, taking out 4 cm×4 cm placental tissue, put it into sterile saline containing antibiotics (anti-penicillin and anti-streptomycin). Bring it into a sterile room, saline flush 5-8 times, using a pair of sterile scissors to cut it into tissue fragments of 1 mm×1 mm in size to operate on readily, add 0.25% trypsin/EDTA, digest at 37 degrees for 15-20 minutes during the digestion process, vortex once every 4-5 minutes. Terminating the reaction with medium containing fetal bovine serum, filter through a 200 mesh sieve, the cells are collected and set aside.
\textcircled{4} OP9, OP9-DL1 recovery, culture and passage. Remove the stored frozen OP9, OP9-DL1 from liquid nitrogen, fast thaw within 2-3 minutes, put it into a Petri dish to incubate in the medium of α-MEM containing 20% fetal bovine serum with super quality. Medium was changed once every three days. When the cells grow to the state of 80-90% fusion, digest them with 0.25% trypsin/EDTA for 4-6 minutes, until the cells deformed, shaking with a gentle force, the cells will be detached from the dish. Terminate the reaction with α-MEM medium containing fetal bovine serum, centrifuge and collect the cells passage at the ratio of 1:3, or set aside.

4. Preparation of three-dimensional cell culture matrix or cell culture scaffolds.

According to the instructions provided by the manufacturer, process the three-dimensional cell culture material, including hydrogels, seaweed, nano-materials. Taking Beaver Nano™ 3D cell culture hydrogel as a representative, elaborate three-dimensional hydrogel material preparation.

The main ingredient of BeaverNano™ hydrogel is polypeptide biological nanomaterial. Around PH of 7.0, it has the ability of self-assembling into network of nano-dimensional scafford with aperture of 50-200 nm, which is similar to the natural form of the extracellular matrix (ECM), providing cell adhesion, differentiation or proliferation with an environment that is much closer to the body's internal situation. The hydrogel has very good biocompatibility, its degradation products are natural amino acids, excluding the existence potential contaminants or pathogenic factors. The detailed protocols for use are as follows:

\textcircled{1} Treat hydrogel with ultrasonic water bath (or Vortex mixer) for 30 minutes to reduce the viscosity of the solution of hydrogel.
\textcircled{2} Use 1% (weight/volume:w/v) solution of hydrogel and 20% (w/v) sterile sucrose solution to make up 2× original hydrogel working solution. Set aside.

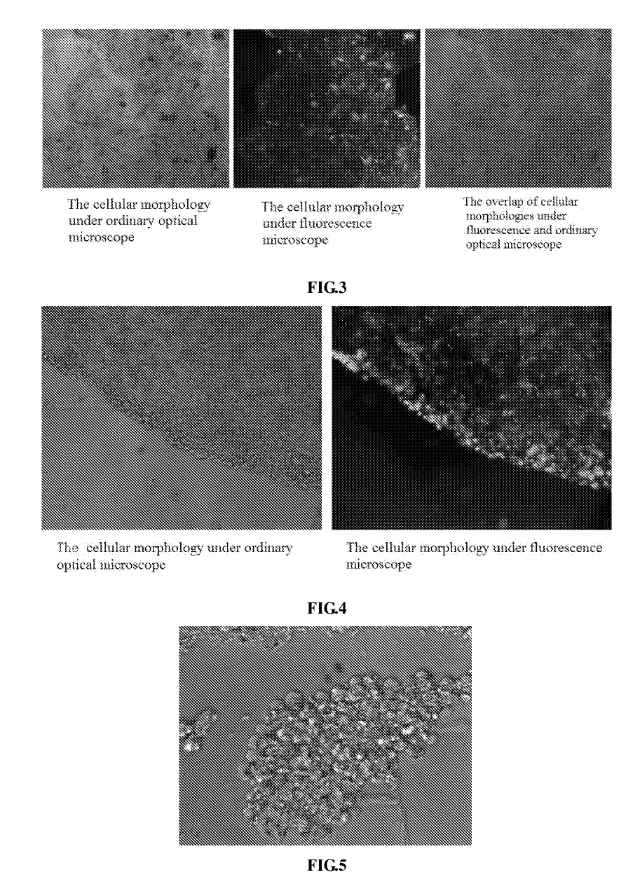

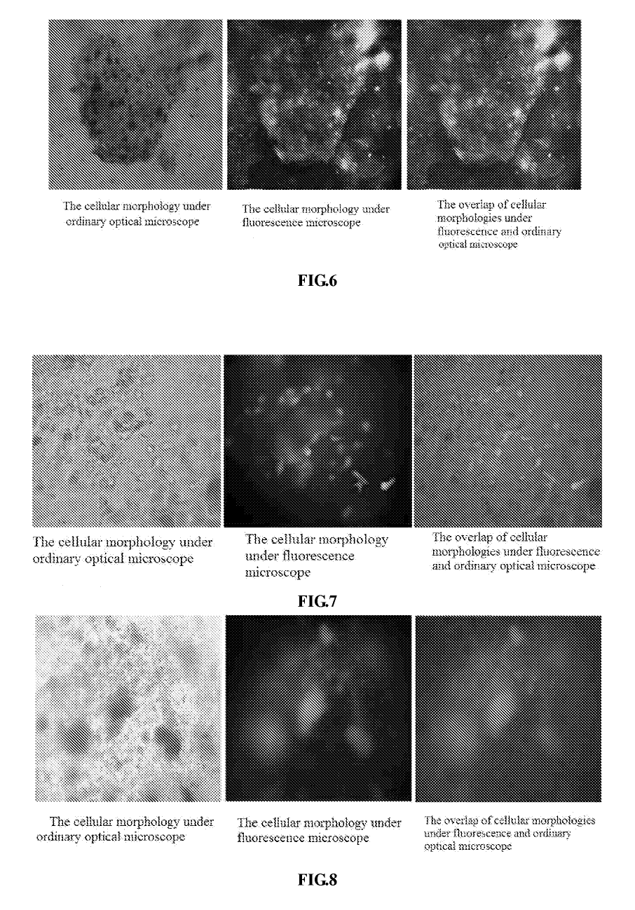

5. Using three-dimensional cell culture matrix or scaffold to establish a system that induce pluripotent stem cells to differentiate into hematopoietic stem cells efficiently.
\textcircled{1} Digest pluripotent stem cells with collagenase IV or mechanical method. In the experimental group of associating stromal cells, trypsin was added to digest OP9, OP9-DL1 and other target cells, after centrifugation, the supernatant was discarded. Pluripotent stem cells and stromal cells were mixed and the target cells are resuspended with sterile 10% (w/v) sucrose solution. The cells are collected after centrifugation.
\textcircled{2} The cells are resuspended at an appropriate amount in sterile 20% sucrose in the working solution, and adjusted to 4×10⁶ cells/ml cell density. The working concentration of 2× formulated into a cell suspension.
   \textcircled{3} Mix equal volume of 2× hydrogel working solution and 2× cell suspension.
   \textcircled{4} Add cell culture medium which contains 15% fetal bovine serum (Hyclone) in IMDM (Iscove's Modified Dulbecco's Mediums) into the upper part of the gel gently along the edge of the culture well, the salt irons of the cell culture medium will lead hydrogel to self-assemble into hydrogel with the fibrous structure.
   \textcircled{5} The plates were placed in an incubator at 37° C. for 30 to 60 minutes, to let them solidify.
   \textcircled{6} During the solidification of hydrogel, carefully remove ⅔ to ¾ of the volume of the culture solution with a 200 μL ranged pipetman, followed by replacement of the culture solution again, and changed twice within 30 minutes.
   \textcircled{7} In the experimental group contained inducing factor, adding the appropriate inducing factor in the medium. At the first stage, the induction medium was IMDM containing 10 ng/ml BMP4, 100 ng/ml SCF, 100 ng/ml FLT3L, 20 ng/ml TPO, 1 ng/ml VEGF, 2 μM PGE2, 15% fetal bovine serum, after induction for 5-7 days. Then switch to the culture solution of the second stage, hematopoietic stem cell expansion medium SFEM (STEMCELL TECHNOLOGY) containing 100 ng/ml SCF, 100 ng/ml FLT3L, 20 ng/ml IL-3, 20 ng/ml IL-6, 20 ng/ml TPO, ing/ml VEGF, 2 μM PGE2). 10-14 days later, the culture medium was replaced for the third stage, which was IMDM, containing 20 ng/ml SCF, 20 ng/ml IL-3, 20 ng/ml IL-6, 20 ng/ml GM-CSF, 20 ng/ml TPO, 20 ng/ml EPO, 10 ng/ml IL-7, 10 ng/ml IL-2, 10% fetal bovine serum, and further cultured to 21-28 days. Throughout the induction process, observe changes in cell morphology of induction system every day. Please refer to FIGS. 3 to 9 for the changes of cell morphology, which show the dynamic changes in cell morphology of the day 0, to fourteenth day, the twenty-eighth day in the three-dimensional guidance system respectively.

6. Use flow cytometry to detect the expression of human cells in three-dimensional system (taken TRA-1-85 as the detection marker) and CD34+ hematopoietic stem cells.

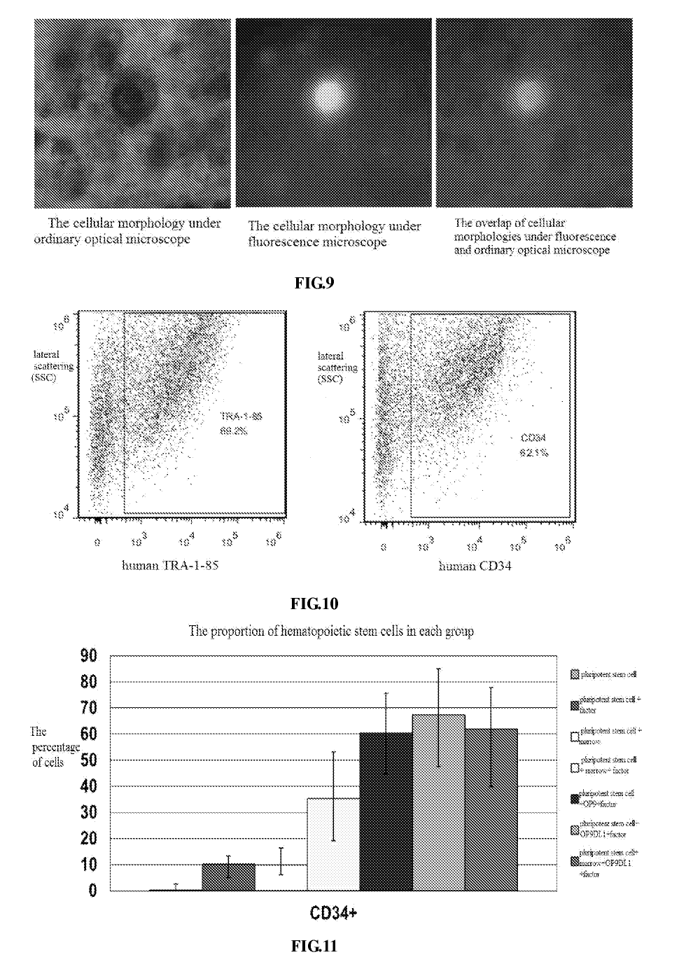

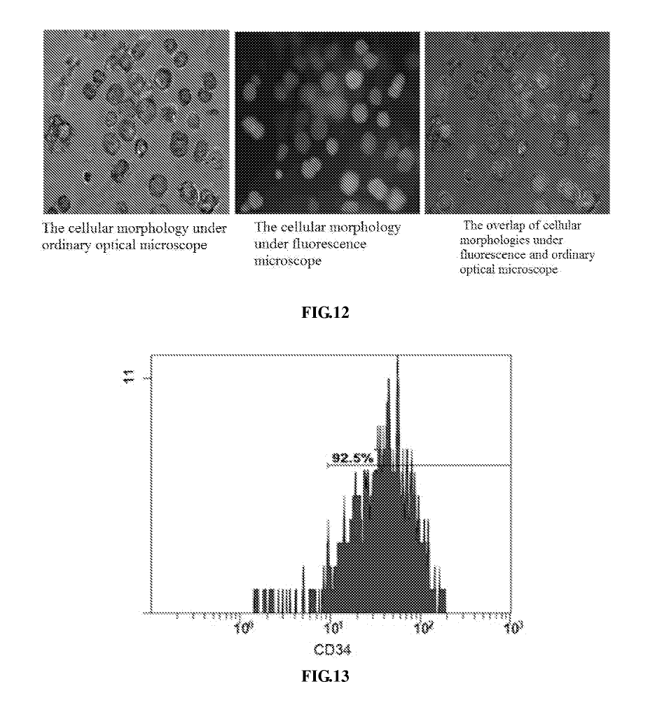

Specific methods are as follows:
\textcircled{1} Collect disposed three-dimensional cultured cells in each group in a phased manner, treat three-dimensional material gently with pipetting tips, cells are collected and subjected to 0.25% trypsin/EDTA digestion for 4-5 minutes, after gently pipetting, fetal bovine serum medium is added to terminate digestion, then filter through 200 mesh sieve.
\textcircled{2} wash 2-3 times with PBS (1% FBS and 1 mM EDTA was added for cell nutrition and to prevent cell adhesion) which does not contain Ca²⁺ and Mg²⁺ to, centrifuge at the speed of 1000 rpm for 5 minutes. The cells are collected, then prepare single cell suspension, the cell density is adjusted to 106-107 cells/ml.
\textcircled{3} In each experimental group, take 20 μl cell suspension, add 5 μl corresponding mouse anti-human antibodies, including TRA-1-85-PE, CD34-PE-Cy5, incubate in dark place at room temperature for 30 min, take isotypic IgG as the control. Use PBS (1% FBS and 1 mM EDTA was added) which does not contain Ca²⁺ and Mg²⁺ to wash 3 times, 500 μl PBS (1% FBS and 1 mM EDTA was added) is used to resuspend the cells. Utilize BD FACScalibur instrument (Becton Dickinson) to detect the expression of surface antigens on human cells and a variety of blood cells including hematopoietic stem cells in the three-dimensional induction system.
\textcircled{3} The obtained data are analyzed using FlowJo Version 7.2.5 software, three batches of corresponding samples are processed. Please refer to FIG. 10 to FIG. 11, which show that the expression of TRA-1-85+ and CD34+ cells in the system of hydrogel three-dimensional cell culture material combined with mouse bone marrow, OP9DL1 and cytokine.

7. Enrichment of hematopoietic stem cells

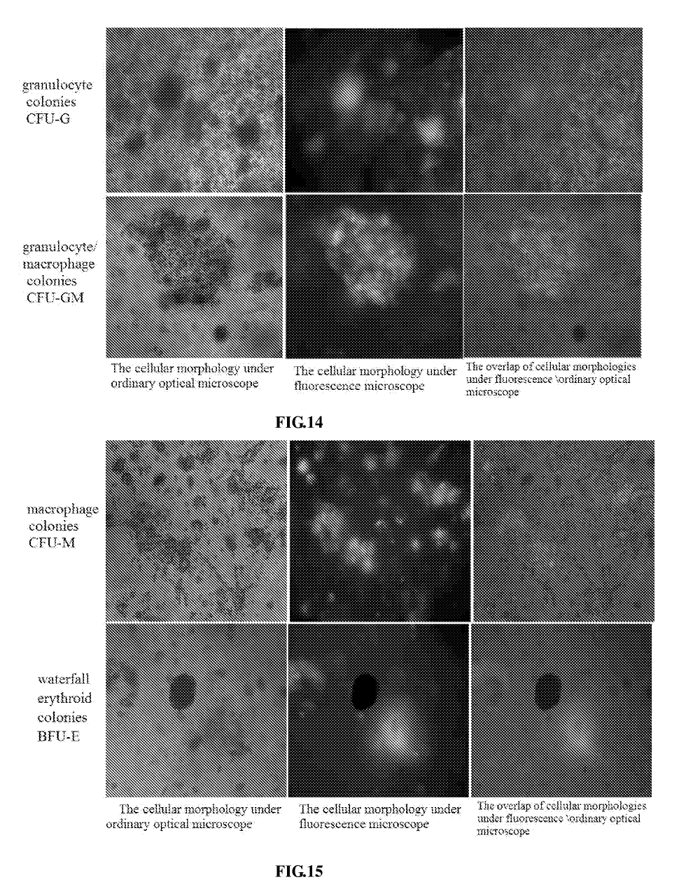

The enrichment of hematopoietic stem cells in three-dimensional inducing system is to use EasySep human CD34 sorting kit from the Canadian Stem Cell Technology Co., Ltd. (STEMCELL Technologies). Because the pre-sorting by flow cytometry shows that the starting percentage of CD34 induction system >20%, the sorting steps had been optimized as follows:
\textcircled{1} Collect cells in three-dimensional cell culture system after 10-14 days induction, gently pipette the three-dimensional material with the tip, centrifuge at the speed of 1200 rpm for 6 minutes to collect the cells.
\textcircled{2} Digest the cells with trypsin solution containing 0.25% EDTA for 5-6 minutes, gently pipetting, use medium containing fetal bovine serum to stop the digestion, filter through 85-mesh sieve.
\textcircled{3} Centrifuge at 1200 rpm, for 6 minutes to collect the cells.
\textcircled{4} Use PBS (1% FBS and 1 mM EDTA is add in) which does not contain Ca2+ and Mg2+ to wash 2-3 times.
\textcircled{5} The cells density is adjusted to 1×10⁸ cells/ml.
\textcircled{6} According to the ratio of 200 uL/mL, add antibody from EasySep CD34 sorting kit.
\textcircled{7} Incubate at room temperature for 15 minutes.
\textcircled{8} Add magnetic beads at the ratio of 100 microliters/ml cell.
\textcircled{9} incubate at room temperature for 10 minutes.
\textcircled{10} Dilute the cells to 2.5 ml with buffer after incubation, put it in magnetic poles, perform a ten minutes' sorting first, and then perform two five minutes' sorting through the poles.
\textcircled{11} Proceed the detection by flow cytometry according to step (4), examine the cells morphology and the expression of CD34. Detect the label of phycoerythrin (PE) on the CD34 antibody clone with coded No. 8G12. FIG. 12 is cell morphology detection after sorting, FIG. 13 is the purity of hematopoietic stem cell sorted by flow cytometry.

8. Detection of the potential of the acquired CD34 cells in three dimensional inducing system to generate hematopoietic colonies (CFU) on methylated cellulose semi-solid culture.

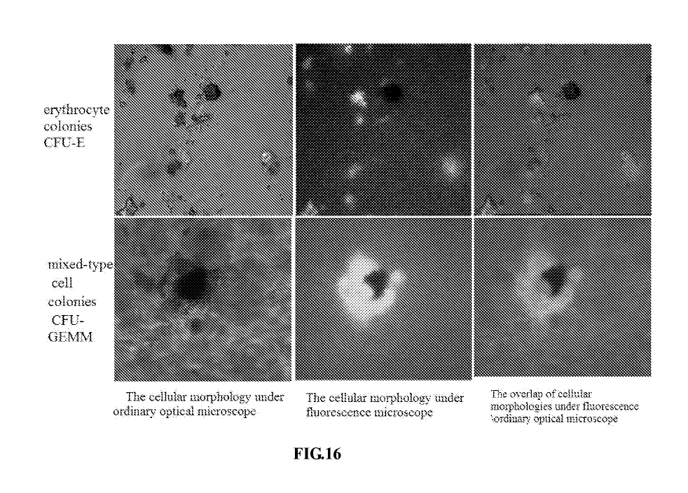

\textcircled{1} After the sorting of CD34 cells, plant them in low cohesive petri dishes which each contain a medium of 9% methylated cellulose, 0.1 mM 2-mercaptoethanol, 2 mM GlutaMAX, 20 ng/mL SCF, 20 ng/mL IL-3, 20 ng/mL IL-6, 20 ng/mL G-CSF, 20 ng/mL GM-CSF, 20 ng/mL TPO, 3 U/mL EPO in Iscove's Modified Dubecco's Medium (IMDM) in accordance with 5000 cells/ml, Continue to culture for 12-14 days.
\textcircled{2} Observe colony formation. After 14-17 days' induction, culture systems have significant colony formation. In terms of the shape of the colonies, the size of the cells, the visibility of cell density, estimate, detect and compare the various hematopoietic cell colony formation, and count. Please refer to FIGS. 14-16 for the result. FIG. 14 shows that CD34 cells sorted in three-dimensional inducing system generate granulocyte/macrophage hematopoietic colony in methylated cellulose semi-solid medium containing hematopoietic growth factors after culturing for 14-17 days. FIG. 15 shows that CD34 cells sorted in three-dimensional inducing system generate megakaryocytic, erythropoiesis hematopoietic colony in methylcellulose semi-solid medium containing hematopoietic growth factors after culturing for 14-17 days. FIG. 16 shows that CD34 cells sorted in three-dimensional inducing system generate erythroid, myeloid/erythroid/macrophage/megakaryocyte colony in methylcellulose semi-solid medium containing hematopoietic growth factors after culturing for 14-17 days.

What is claimed is:

1. A method of utilizing three dimensional inducing system to obtain hematopoietic stem cell (HSC), wherein, the method is realized by the following technical solutions: use three dimensional inducing system, according to different functions of cytokines, induce pluripotent stem cells to differentiate into hematopoietic stem cells with high efficiency in a phased manner, gain more than 40% hematopoietic stem cells after 10 to 14 days' induction and amplification, said pluripotent stem cells include human embryonic stem cells and induced pluripotent stem cells, said three dimensional inducing system selected from multiple three-dimensional cell culture medium or cell culture scaffolds.

2. The method of utilizing three dimensional inducing system to acquire HSC of claim 1, wherein, said multiple three-dimensional cell culture medium or cell culture scaffolds are three-dimensional cell culture material made from a variety of hydrogel, seaweed material.

3. The method of utilizing three dimensional inducing system to acquire HSC of claim 1, wherein, said selected cytokines at the first stage are BMP4, SCF, Flt3L, VEGF, TPO or PGE2, said selected cytokines at the second stage are haemopoietic factors and the group which promote the multiplication of hematopoietic stem cells such as SCF, Flt3L, IL-3, IL-6, TPO, VEGF, PGE2, said selected cytokines at the third stage are the group which promotes amplification and differentiation of hematopoietic cells such as SCF, IL-3, IL-6, IL-7, IL-2, GM-CSF, EPO, TPO.

4. The method of utilizing three dimensional inducing system to acquire HSC of claim 1, wherein, said human embryonic stem cells are embryonic stem cell lines which are commercially available, induced pluripotent stem cells are those acquired by inducing human cells, using retrovirus, lentivirus, adenovirus or sendai virus which containing transcriptional factor such as Oct 4, ox2, Klf4 and\or c-Myc.

5. The method of utilizing three dimensional inducing system to acquire HSC of claim 1, wherein, stromal cells are mixed in three-dimensional cell culture medium or cell culture scaffolds, said stromal cells are human bone marrow cells, mouse bone marrow cells, placenta cells, mouse bone marrow cell line OP9, OP9-DL1 or other related stromal cells which induce the differentiation of hematopoietic cells.

6. The method of utilizing three dimensional inducing system to acquire HSC of claim 1, wherein said three dimensional inducing system are three-dimensional cell culture medium or cell culture scaffolds and induced differentiation factors and growth factors.
